(19) United States
(12) Patent Application Publication
(10) Pub. No.: US 2020/0073560 A1
(43) Pub. Date: Mar. 5, 2020

(54) METHODS FOR DECENTRALIZED GENOME STORAGE, DISTRIBUTION, MARKETING AND ANALYSIS

(71) Applicant: Genetic Intelligence, Inc., New York, NY (US)

(72) Inventor: Bertrand T. Adanve, New York, NY (US)

(21) Appl. No.: 16/559,263

(22) Filed: Sep. 3, 2019

Related U.S. Application Data
(60) Provisional application No. 62/726,725, filed on Sep. 4, 2018.

(51) Int. Cl. G06F 3/06; G16B 50/40; G16B 40/00; G16B 50/30; G06F 16/907; H04L 9/32; H04L 9/06

(52) U.S. Cl. CPC G06F 3/0622; G06F 3/0644; G06F 3/067; G16B 50/40; H04L 9/065; G16B 50/30; G06F 16/907; H04L 9/3213; G16B 40/00

(57) ABSTRACT

Techniques for storing omics data that indicates long sequences of elements associated with a particular biological molecule include receiving digital omics data comprising over two kilobytes. The digital omics data is split into multiple partitions. The maximum partition size is much less than then a number of elements in a typical instance of the particular biological molecule. Each partition is encrypted. Each encrypted partition is inserted into a corresponding data packet that includes an owner field that uniquely indicates an owner of the omics data. Each data packet is uploaded into a non-centralized, peer-to-peer distributed storage network. Thus, genome data is encrypted and stored in a distributed, scalable, fully decentralized, fast, and highly secure network. The network further provides for decentralized computing, trustless validation of the genomes by way of oracles and trustless genome analysis by third party providers through smart contracts.

### CROSS-REFERENCE TO RELATED APPLICATIONS

This application claims benefit of Provisional Appln. 62/726,725, filed Sep. 4, 2018, the entire contents of which are hereby incorporated by reference as if fully set forth herein, under 35 U.S.C. § 119(e).

### FIELD OF THE INVENTION

This invention relates to systems and methods for storing, distributing, marketing, retrieving, and analyzing genomes (and other omics data) in a scalable and trustless decentralized fashion.

### BACKGROUND OF THE INVENTION

In the following discussion, certain articles and processes will be described for background and introductory purposes. Nothing contained herein is to be construed as an “admission” of prior art. Applicant expressly reserves the right to demonstrate, where appropriate, that the articles and processes referenced herein do not constitute prior art under the applicable statutory provisions.

The genomics revolution has regained vigor in recent years aided by the sharp drop in genome sequencing cost and improved access to big data computing and storage, often by cloud infrastructures. Ever more efficient sequencing machines are generating vast swaths of genome data on an increasing portion of the population (e.g., with every new baby being sequenced at birth and genetic sequencing incorporation into the routine diagnostic at the hospital). This unique marriage of life sciences and technology points to a future world where genome data can be leveraged to improve people's lives by accelerating the discovery of causative genetic features underlying disease and creating better medicines and diagnostics.

However, this tremendous future promise is threatened by issues of security. Genome data must be held somewhere that is secure and prioritizes the data owner's rights over the data itself; yet easy transfer and sharing of the data is necessary for researchers to analyze and generate benefits such as diagnostics and treatments. This dilemma between facilitating research through more fluid and rapid data sharing and protecting owner privacy and data rights is certainly a challenge. Data security has revealed itself as one of the greatest problems of the digital age in the face of repeated data hacks from servers and their leak or sale on black markets by cyber criminals. Malicious agents are perfecting ever more sophisticated hacking techniques that target the private data of the general populace, and even the largest organizations have been hacked, despite their implementation of security protocols. For instance, hackers breached three billion accounts at Yahoo (Larson, CNN News. (2017)), 83 million accounts at JPMorgan Chase (Bernard, The New York Times. (2014), and 143 million accounts at Equifax (Lieber et al., The New York Times. (2017). With more and more data being held by individuals or corporations on databases or servers connected to the internet, data breaches have become the norm rather than the exception.

Further, if people's personal genome data are centralized in the hands of a single party as has often become the case with other personal data types, sharing will be inhibited and reserved to a select few that align with the centralizing party's motives. A conflict of interest may develop between this centralizing party prioritizing its own bottom line ahead of all else and the data owners desiring personal benefit from contributing their data. In addition, these centralizing parties constitute data moats (and honey pots to hackers) that extract extremely high rent for their service, take control away from the primary data owner (e.g., most personal genomics companies require patients to fully give up their ownership of the data), and introduce friction into the network. Centralization of data is also vulnerable to other failure modes such as natural disasters and large hardware malfunctions that can wipe out entire databases permanently.

Accordingly, there is significant need for a system where genome data is safe and secure, yet does not require reliance on a centralizing third party, and control of the data remains in the hands of the primary genome data owner to guardedly share with whichever party they choose, given the right incentives. The present invention addresses this need and provides novel compositions and methods that have not previously been disclosed in the art. Features, such as secure storage, transaction, and transmission of data in a decentralized network and system robustness in the face of natural disasters and hardware malfunctions, are discussed.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings, and as set forth in the examples and appended claims.

The present invention provides tools, systems, and methods for the storage, marketing, distribution, retrieval, and analysis of genomes (and other omics data) in a trustless, decentralized manner. Specifically, the present invention provides methods that facilitate vast, decentralized networks where genomes are readily available but are invulnerable to attack by nefarious parties. On these networks, provided by the disclosed methods, vendors can make available genomic analysis tools and applications that help a person to better understand their genomic profiles, health risks, etc. while still maintaining such critical information private to only that person (i.e., genome or omics data owner) or an authorized representative.

The present disclosure describes how to implement such a secure and decentralized network for genome (and other omics) data storage, processing, marketing, distribution, retrieval, and analysis. Specifically, the methods provide for a network platform that enhances security and accessibility by obviating centralized systems which are replete with inefficiencies and risks including third party abuse of data or rights, lack of transparency, and single points of failure.

Researchers are often dependent on data owners to share their data for analyses. Greater privacy and ownership rights afforded to data owners would encourage them to make their data available to researchers on the network, which would stimulate research findings for better health and phenotypic outcomes. Furthermore, data sharing can be encouraged by proper incentivization, including the use of monetary incentives.

This network for decentralized storage and marketplace is aimed to service genome (and other omics) data as well as their attendant services or applications. Network security is maintained using implementing tamper-resistant or tamper-proof encryption of packets of data. These encrypted data undergo unpredictable, unmonitored, and extensive re-routing of encrypted packets of data, which provides assurance against tampering and unauthorized access.

In one set of embodiments, a method is executed on a processor for storing omics data that indicates long sequences of elements associated with a particular biological molecule. The method includes receiving digital omics data comprising over two kilobytes (KB, 1 KB=1024 bytes, each byte equal to 8 bits). The method also includes splitting the digital omics data into multiple partitions, each partition holding a number of elements in a sequence of the omics data. The number of elements is not greater than a maximum partition size; and, the maximum partition size is much less than then a number of elements in a typical instance of the particular biological molecule. The method further includes encrypting each partition to form an encrypted partition. Still further the method includes inserting each encrypted partition into a corresponding data packet that includes an owner field that holds data that uniquely indicates an owner of the omics data. The method even further includes uploading each data packet into a non-centralized, peer-to-peer distributed storage network.

In some embodiments of this set, the maximum partition size is in a range from 0.5 megabytes (MB, 1 MB=1024 KB) to 10 MB and preferably in a range from 0.5 MB to 1.5 MB. In some of these embodiments, the digital omics data comprises more than 0.5 MB and preferably more than 10M B.

In some embodiments of this set, the number of elements in each partition is not less than a minimum partition size and the minimum partition size is in a range from 0.5 KB to 3 KB and preferably in a range from 0.5 KB to 1 KB.

In some embodiments of this set, the owner field is encrypted. In some embodiments of this set, the corresponding data packet includes a token field that holds data that indicates a means to pay for a service that operates on the encrypted partition.

In some embodiments of this set, the corresponding data packet includes a sequence field that holds data that indicates a position of the encrypted partition in the data packet relative to a different partition in the digital omics data. In some of these embodiments, the sequence field is encrypted.

In some embodiments of this set, the omics data includes data about one or more biological molecule types selected from a group comprising genomes, proteomes, kinomes, phenomes, epigenomes, metabolomes and transcriptomes.

In some embodiments of this set, each data packet is stored on a plurality of different nodes on the distributed storage network. In some of these embodiments, each different node of the plurality of different nodes on which each data packet is stored is in different geographical regions.

In some embodiments of this set, the method yet further includes compressing each partition before said encrypting; or, compressing each encrypted partition after said encrypting; or, both.

In some embodiments, the corresponding data packet includes a metadata field that holds data that indicates descriptive information about the partition. In some of these embodiments, the metadata field is encrypted.

In some embodiments of this set, said encrypting is homomorphic encrypting relative to one or more operations on the encrypted partition.

In some embodiments of this set, the method includes granting permission to a second party different from the owner of the omics data to decrypt a field in the data packet. In some of these embodiments, granting permission to the second party is performed in response to receiving a payment. In some of these latter embodiments, the payment is in a form of a network token. In some of these embodiments, the method includes receiving an analysis result based on the field decrypted in the data packet in response to granting permission to the second party. In some of these embodiments, receiving the analysis result is performed in response to sending a payment. In some of these latter embodiments, the payment is in a form of a network token. In some of these embodiments, granting permission to the second party is facilitated automatically using a smart contract.

In some embodiments receiving the analysis result includes inserting, into an analysis field in the data packet, data that indicates the analysis result.

In some embodiments of this set, a catalogue of available omics data from one or more owners of the omics data is maintained on the distributed storage system.

In some embodiments granting permission, the multiple digital omics data are used by the second party.

In other sets of embodiments, a computer-readable medium, or apparatus or system is configured to perform one or more steps of one or more of the above methods.

In one preferred implementation, the genome (or other omics) data are locally cut into smaller partitions or segments of data, encrypted on the owner's local compute device, and then uploaded onto a network for distributed storage and peer-to-peer communication (e.g., SAFE Network, Holochain) to be accessed at a later time by the data owner or by a party to whom the data owner has granted the requisite permissions.

In one preferred implementation, two or more copies, but preferably at least three or more copies of uploaded genome (or other omics) data are copied onto different servers or nodes in the network to ensure data preservation. These copies are stored randomly across the network's computer devices (nodes) or, in some implementations, required to be stored on at least two or more different physical regions (e.g., continents) or other designations of geographical location, which will increase network resistance to natural disasters, hardware malfunctions, and other localized or even widespread failures.

In one implementation, a decentralized group consensus mechanism is used to create essentially randomly chosen nodes to be grouped together and validate an event.

In other implementations, nodes can be economically incentivized to perform their tasks based on various functions that scale differently with size of node or number of events handled.

In other aspects, the genome or other omics data is first compressed before processing for secure and efficient upload onto the network.

In other aspects, the network or service providers can optimize the storage capacity storage on the network by merging segments of identical data between different uploaded genome or other omics data sets and revising preceding segments to call on these merged segments. An AI protocol can be employed to this end.

In other aspects, the data can be uploaded in various states of data processing (e.g., for a genome, these data structures include raw FastQ, Bam, VCF). In certain implementations, these data can be converted between different data structures on the network.

In other implementations, anonymous and secure links can be established between the omics data on the network to achieve massive arrays of data (FIG. 4) that will facilitate discovery of biomarkers (e.g., genetic features) that underlie various phenotypes (e.g., Alzheimer's disease).

In other aspects, oracles are employed to autonomously confirm and validate the descriptive information appended to the genome (or other omics) data (e.g., phenotypic information associated with genome data).

In other aspects, oracles are employed during upload, storage, analysis, or data processing to detect quality of data or duplication of data. For example, oracles can assess genome data for extent of coverage or completeness. Oracles can implement these analyses with or without needing to decrypt the data and using tools such as cryptographic hash functions or genome fingerprinting. The analyses can be encrypted and either appended to the genome data or transmitted directly to the requesting party.

In yet other aspects, the network harbors actors that provide omics data services to data owners on the network including analysis, interpretation, diagnostics, and health counseling, all privately and securely and with the genome owner always retaining full control of their data.

In one implementation, a single reputation system or multiple reputation systems for genome and other omics service providers, including, but not limited to, providers of storage, marketing, and analysis is used to determine reliability, accuracy, and trustworthiness of service. A single reputation system or multiple reputation systems can also be implemented to establish consensus among resource providers and across the overall network. These reputation systems may be based on a combination of factors, including, but not limited to, age, reliability or uptime, or a user-based rating system. The calculation of a reputation factor can be performed using set formulae or through learning algorithms supported by AI.

Furthermore, the identity of the data owner always remains known only to the data owner alone, ensuring fully privacy during any exchange on the network, when providing data or acquiring services. Other parties such as those providing omics or network services can also maintain anonymity or choose to reveal their identity.

In yet other aspects, smart contracts are employed to automatically execute services between various parties on the network such as validation, analyses, negotiation, distribution, marketing, etc. (FIG. 5).

In other implementations, bundling of services or data can be accomplished autonomously by the network.

In certain implementations, allocation of available network computing power for various services can also be automated.

In specific implementations, the systems and methods of the disclosure can utilize a decentralize computation layer to outsource computation intensive tasks to qualified nodes in a trustless fashion.

In certain implementations, the network can use AI protocols for analysis of genome or other omics data. The computational power of the network can be leveraged to run many AI simulations on data in parallel.

In other implementations, an AI can be used to predict, implement or establish services on the network.

In other implementations, the network comprises the use of a token to incentivize and/or facilitate the exchange of genome data and services.

In other implementations, the network is fully autonomous with no human intervention to determine the cost of storage or service, rather, these prices being set automatically by the network based on calculated resource availability. These calculations can be optimized with AI-enhanced learning algorithms.

In yet other implementations, different omics data sets (e.g., kinome, proteome, phenome, transcriptome, etc.) are uploaded to the network to be stored, processed, validated, analyzed, or marketed in a synergistic or otherwise fashion to the genome or with each other.

In another implementation, the genome data is stored on the owner's local machine and transferred through an encrypted, direct, peer-to-peer gateway (e.g., WebRTC) after the requisite negotiation and permissions have been achieved through a scalable, decentralized network (e.g., IOTA, SAFE Network, Holochain, etc.).

An example, simplified work flow for specific systems and methods of the disclosure is illustrated in FIGS. 1-5.

These and other aspects, features and advantages will be provided in more detail as described herein.

### BRIEF DESCRIPTION OF THE FIGURES

FIG. 1 is a schematic diagram illustrating a simplified view of the system of various interacting layers of the scalable, secure, and decentralized omics data services network provided by and used by peer-to-peer compute devices.

FIG. 2 is a simplified schematic view of a representative omics data file as it is cut into chunks, encrypted and stored as many persisting and randomized copies across storage nodes on the decentralized network.

FIG. 3 is a schematic representation of the blockchain versus the more scalable DAG-based structure and consensus.

FIG. 4 is a schematic diagram illustrating an example library to connect front end apps and services to underlying securely, de-centrally stored and semantically connected omics data without abridging data owners' control of the data.

FIG. 5 depicts a schematic diagram illustrating smart contracts facilitation of automated exchange of data and services between participants throughout the network.

FIG. 6 depicts a compute device. Displayed distributed compute and distributed storage instances are provided remotely through the distributed network.

### DETAILED DESCRIPTION

The following description is presented to enable one of ordinary skill in the art to make and use the invention and is provided in the context of a patent application and its requirements. Various modifications to the example embodiments and the genetic principles and features described herein will be readily apparent. The example embodiments are mainly described in terms of particular processes and systems provided in particular implementations. However, the processes and systems will operate effectively in other implementations. Phrases such as “example embodiment”, “one embodiment” and “another embodiment” may refer to the same or different embodiments.

The example embodiments will be described with respect to methods and compositions having certain components. However, the methods and compositions may include more or less components than those shown, and variations in the arrangement and type of the components may be made without departing from the scope of the invention.

The example embodiments will also be described in the context of methods having certain steps. However, the methods and compositions operate effectively with additional steps and steps in different orders that are not inconsistent with the example embodiments. Thus, the present invention is not intended to be limited to the embodiments shown, but is to be accorded the widest scope consistent with the principles and features described herein and as limited only by appended claims.

It should be noted that as used herein and in the appended claims, the singular forms “a”, “and”, and “the” include plural referents unless the context clearly dictates otherwise. Thus, for example, reference to the effect of “a network” may refers to one or a combination of networks, and reference to “a method” includes reference to equivalent steps and processes known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those limits are also included in the invention.

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. All publications mentioned herein are incorporated by reference for the purpose of describing and disclosing the formulations and processes that are described in the publication and which might be used in connection with the presently described invention.

Those skilled in the art will recognize, or be able to ascertain using no more than routine experimentation, many equivalents to the specific embodiments of the invention described herein in the detailed description and figures. Such equivalents are intended to be encompassed by the claims.

For simplicity, in the present document certain aspects of the invention are described with respect to use of certain methods. It will become apparent to one skilled in the art upon reading this disclosure that the invention is not intended to be limited to a specific use, and can be used for in a wide array of implementations including secure genome storage, marketing, distribution, retrieval and analyses services (e.g., ancestry, diagnosis, prognosis, etc.).

### Definitions

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “genome”, “genetic sequence”, and “nucleic acid sequence” as used interchangeably herein refers to any sequence data obtained from nucleic acids from an individual of any species (e.g., H. sapiens). Conventionally, genome refers to the complete set of nucleic acid sequences for an individual of a species. Such data includes, but is not limited to, whole genome sequencing data, exome sequencing data, transcriptome sequencing data, cDNA library sequencing data, kinome sequencing data, metabolome sequencing data, microbiome sequencing data, single nucleotide polymorphism determination, targeted sequencing data, microarray data, epigenome data, and the like.

The term “omics” refers to a biological study performed on a wide scale encompassing the elements in the specific biological category. For instance, proteomics is the study of the proteome, transcriptomics that of the transcriptome, genomics that of the genome, and metabolomics that of the metabolome. “Omics data” thus refers to data representing the various biological categories and their associated elements. The omics data has in common long sequences of elements associated with a particular biological molecule.

The term “genetic features” as used herein includes any feature of the genome, including sequence information, epigenetic information, etc. that can be used in the methods and systems as set forth herein. Such genetic features include, but are not limited to single nucleotide polymorphisms (“SNPs”), insertions, deletions, codon expansions, methylation status, translocations, duplications, repeat expansions, microsatellites, rearrangements, copy number variations, multi-base polymorphisms, splice variants, etc.

The terms “decentralized” or “decentralization” refer to the organization, reorganization, distribution, or redistribution of people, functions, processes, authority, and so forth without recourse to a central authority.

The term “DLT” or “distributed ledger technology” is a database infrastructure that allows for identical and immutable recording, access, validation and upkeep by a number of independent participants (i.e., nodes) spread across different geographic locations. A DLT can be decentralized or centralized (i.e., a majority of the nodes in the network are controlled by a single party or nodes require a centralized party's permission to participate).

The term “trustless” refers to a state that obviates the need to understand the motives, intent, or trustworthiness of another party to enter into exchange with them. Traditionally, the cost of trust in human society has been high as structures, laws, and companies have been erected to ensure the validity of exchanged goods and transactions. For instance, financial institutions are the arbiters that exchanging parties put their trust in to perform a monetary transaction in exchange for a fee or credit rating agencies like Equifax are the centralized arbiters that are consulted regarding a party's credit worthiness. There are methods to automate trust so that each participating party in an exchange can easily verify validity of any exchange without having to rely on a central party at huge cost.

The term “smart contract” refers to a compute device function that executes automatically when it registers that the conditions of the exchange agreement between two or more transacting parties have been met.

The term “oracle” refers to the providers of data and/or services from the outside world that feed and potentially trigger smart contracts on decentralized networks.

The term “QUIC” (Quick UDP Internet Connections) refers to a transport layer network protocol standard designed to have improved latency performance compared to protocols such as TCP. It further incorporates cryptographic handshake thus obviating the need (and inefficiency) for a separate security layer such as SSL.

The term “homomorphic encryption” refers to an encryption method that allows for computation of encrypted data (i.e., ciphertext) such that the result of this computation is identical to the same computational operation performed on a non-encrypted version (i.e., plaintext) of the same data. The result of the analysis is encrypted and can only be decrypted by the owner of the input data.

The terms “artificial intelligence” and “AI” as used interchangeably herein denotes intelligent systems or techniques such as machine learning, artificial neural networks, and the like.

The term “blockchain” refers to an early type of DLT where data packets called blocks, which are events representing transactions, tasks, etc., are recorded chronologically and according to certain strict rules such as specific interval of time or the identity of the recorder (e.g., respectively 10 minutes and by the Proof-of-Work winner in the case of Bitcoin).

The term “Bitcoin” or “BTC” refers to the first decentralized application that popularized the blockchain.

The term “Ethereum” or “ETH” refers to an application of the blockchain that popularized smart contracts and decentralized computing.

The term “directed acyclic graph” or “DAG” refers to a later generation DLT where the ordering of events is per their dependency relationships (i.e., directed arrows or edges) such that events cannot form loops within the network structure and such that every edge is directed from earlier to later, the time flow is in one direction. DAGs can be employed to model many types of information, especially when modeling events' influence on one another. They are an attractive structure to achieve scalability in DLT.

The term “IOTA” or “Tangle” refers to a later generation decentralized application that is based on DAG structure. It further incorporates “Qubic”, which is among other things a decentralized computing layer allowing for smart contracts using functional language programming.

The term “consensus” refers to agreement amongst all participants in the decentralized network on the validity of an event (e.g., a transaction, data access). A consensus mechanism is employed to achieve this end so as to prevent malicious (i.e., Byzantine) actors from disrupting the network. Bitcoin relies upon “Proof-of-Work” (“POW”) as its consensus mechanism, meaning that network nodes compete to solve a cryptographic problem and the winner (which is not known ahead of time) is the one to update the global blockchain ledger for all participants. The Bitcoin blockchain is the global, distributed record of these successive consensus events. There are other types of consensus mechanisms including “Proof of Stake” and “Proof of Resource”.

The term “PARSEC” or “Protocol for Asynchronous, Reliable, Secure and Efficient Consensus” refers to a later generation consensus mechanism. It is highly asynchronous and Byzantine fault tolerant (i.e., guaranteed to reach mathematical certainty despite the presence of malicious actors and imperfect communication).

The term “SAFE Network” refers to the public decentralized data network built by MaidSafe. Safe Network is based upon several protocols including a sharded version of a DHT protocol known as Kademlia for structure, DAG for message relay between nodes, PARSEC for consensus mechanism, a program known as “CRUST” for secure data transmission, and node storage program known as “Vault” for data storage.

The term “WebRTC” refers to a peer-to-peer connection API that is furnished in most modern web browsers for direct, fast, and secure communications and data transfers. A WebRTC connection is preceded by an identification stage between the connecting clients. This stage is typically handled by centralized signaling servers. Then once identification is confirmed, a “STUN” link allows firewall penetration and secure connection between two or more parties. The identification stage can be adapted to employ decentralized network nodes instead to achieve signaling and negotiation between parties.

The term “Holochain” refers to a distributed data protocol.

The term “Hashgraph” refers to a distributed consensus algorithm based on gossip about gossip pioneered by Swirlds™. “Hedera Hashgraph” refers to the distributed network established by the same company based on the Hashgraph consensus algorithm.

The term “node” refers a compute device that participates in a decentralized network. A node can have any number of functions (e.g., observation, storage capacity, transaction processing, computing capacity, events validation, etc.).

The term “compute device” refers to an electronic device that can take in data input, process the input, perform calculations based on the inputs, and/or transmit the information to other compute devices. A compute device typically has several components including processor, programming, memory, and optional display. Some of these components can be directly attached, located locally, or located remotely (e.g., display).

The terms “FastQ”. “BAM”, “CRAM”, and “VCF” refer to genomes and omics file formats. FastQ represents a more raw version of the data file while BAM (aligned file) and VCF (variants) represent more processed versions of the data file.

The term “hash” refers to the value generated by a “hash function” in mapping data of arbitrarily-large size to data with a fixed size. The same data should always return the same hash, with any deviation indicating modifications (e.g., tamper) to the data. Hashes and hash functions are often structured within “hash tables” and leveraged for rapid array-based data lookup. As the name indicates, a “Distributed Hash Table” or “DHT” is a distributed version of a hash table allowing any participating node the ability to retrieve the data associated with a hash among others.

The term “shard”, “sharding” or “sharded” refers to the partitioning of a distributed network into smaller interacting networks that together constitute the original parent network. Distributed or decentralized networks can leverage sharding to achieve greater scaling throughput.

The term “ASICS” is short for an application-specific integrated circuit. In other words, an ASICS is an integrated circuit or chip that is designed and optimized for a particular purpose, which renders it practically useless for any other purpose other than the one designed for.

The term “API” is short for application programming interface.

The term “webhooks” refers to methods to automatically modify the behavior of a web application given a certain trigger or event.

The term “STUN”, short for Section Traversal Utilities for NAT, refers to a protocol for establishing a direct connection across a NAT (network address translator), which in turn is a technique for simplified communication between private and external networks.

The term “Solid” refers to a set of tools and conventions proposed by Prof. Tim Berners-Lee of MIT for decentralized social applications in order to achieve better privacy and data ownership.

### Overview

The present invention provides systems, tools and methods for storing, distributing, marketing, retrieving, analyzing, and leveraging genome data in a completely decentralized, scalable, and private manner. The invention includes a combination of scalable distributed ledger technology methods, network communications, and genome processing/analysis approaches. This combination of tools is a novel approach broadly applicable to any number of healthcare data, especially those data that are of high dimensions or large in size, and including genetic sequences.

In modern society, exchanges between transacting parties are typically effectuated through a third party that is principally charged with verifying the validity of the transaction so that malicious parties cannot take advantage of honest parties. This setup has conferred untold, centralizing powers into these trusted third parties who can abuse those powers and the data entrusted to them. Furthermore, these third parties become honey pots that hackers target, increasingly successfully, to steal or manipulate valuable information. Recent examples of these unfortunate realities include Facebook's selling of users data (Granville, The New York Times. (2018)) and the hacks of Yahoo, JPMorgan Chase. and Equifax for billions of breached datasets.

The vast troves of genetic data that are being generated from the ongoing genomics revolution have so far been subjected to the same setup where a centralized party coalesces people's genome data and controls access to and often claims ownership of that data. These centralized parties may sell access to the data to the highest bidder (e.g., 23&Me sharing of its customers data with Genentech, GSK, etc.) without the true or original data owners' input or stake in the profits generated by the use of their genetic information. These kinds of arrangements, not to mention the hacking risks from the centralized pooling of data, discourage many people from having their genome sequenced or analyzed, because they are rightfully afraid of forever relinquishing control of their most private data, the biological code of who they are as a unique living being.

The tools and methods described herein improve dramatically upon the centralization status quo by detailing a decentralized network where genomes are stored, analyzed, and marketed while providing security, accessibility, preservation, control, and services to the genome data owner. This system offers the respective sought-after benefits to all participants. Utilizers of genome data (e.g., scientific researchers) can mine greater amounts of data to make discoveries and novel products while the true genome data owners maintain their data rights, share in profits from their data being utilized, and benefit from cures and diagnostics that are developed from the enabled research and development.

The advent of Bitcoin's blockchain in 2008 provided an ingenious, automated solution to the critical problem of double spend that required third parties to insinuate themselves between almost all transactional exchanges as the trust purveyors in exchange for high rent. Others have thus turned to blockchain to attempt to fix the described problem of centralized genome data storage (e.g., Zenome, Shivom, LunaDNA, Nebula Genomics, Encrypgen). However, the blockchain is ill-suited for decentralizing genome data storage because the average human genome file is very large (˜100 GB in size), and blockchain is not a scalable technology. For instance, the blockchain is inefficient (every node in the network must store the ledger), very slow (7 transactions per second when several orders of magnitude more are needed for genome storage), limited access (transaction speeds decrease and costs increase with increasing number of users), and tremendously wasteful (POW wastes more energy than entire countries spend yearly). The blockchain is a decentralized technology that can be effective for immutability and censorship-resistance for digital currencies but not for genome storage.

The limitations of the blockchain make the storage of sizeable data on a blockchain infeasible at scale. This reality is why existing attempts at decentralized genome storage have effectively remained centralized on some level. For instance, some existing decentralized genome storage approaches store the hashes of genome variant files onto a blockchain (i.e., pointers to help with identification and tracking), but the genomes themselves are still stored by traditional (directly on the owner's compute device or on a separate storage service) or centralized means. These current methods are still vulnerable to hacks and misuse due to either centralization or the lower security of traditional storage methods.

Importantly, the methods herein described not only provide a superior alternative to existing centralized genome storage and analysis solutions, but they also overcome the limitations of existing blockchain-based decentralized genome storage methods. Genome and other omics data are much more secured against file storage lost, machine failures, and data hacks. Data files are transferred at network speed, and analyses are performed quickly and anonymously (i.e., without revealing the identity of the data owner). Network congestion does not hamper this network, unlike blockchain technologies in which the already slow network speeds slow down further with an increasing number of participants and/or transactions. The herein described methods and tools allow the network and its described components and combinations thereof to scale with increasing number of transactions. Unlike with blockchain, there are no confirmation delays, no mining waste, and no transaction fees.

### A More Robust Genome Preservation

Nature's approach to genome preservation has been to conserve it inside of the organism's cellular nucleus during their lifetime and combine it with another organism of the same species (if sexually reproducing) for preservation across generations. Nature's methods have advantages. However, it would be advantageous to supplement nature's approach with a truly tamper-proof approach that both secures and preserves an organism's genomes far into the future until such time that technology might allow for further health or phenotypic treatments or services whether pre- or post-mortem.

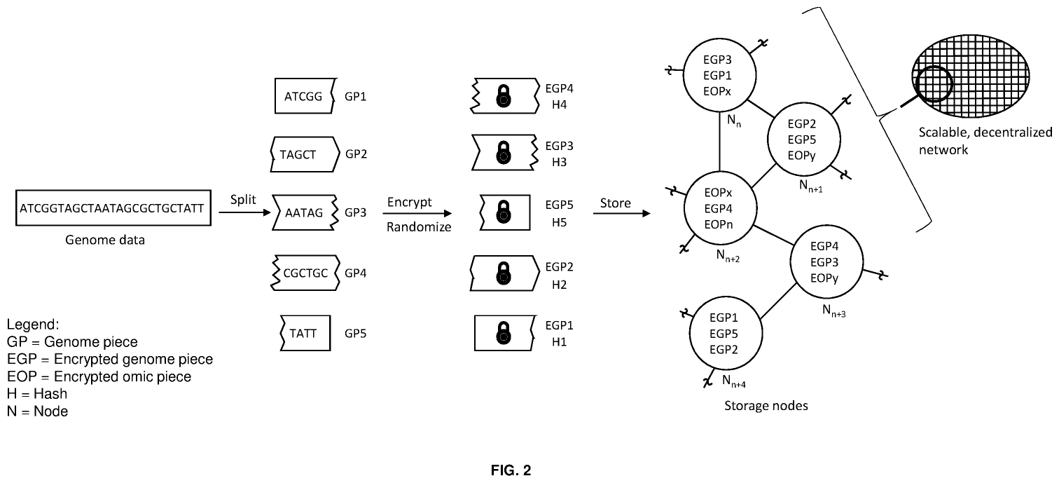

Nature also encrypted living organisms' code of life (i.e., their genomes) until human ingenuity broke that code in 1953. To ensure the genome data's security and privacy, the network provides for the omics data to be fragmented into smaller segments, also called partitions herein, of two or more, which segments are encrypted (e.g., by AES-256 encryption or Winternitz one time signature for quantum computing proof) (FIG. 2). To facilitate network transfer of the pieces, the maximum partition size is in a range from 0.5 MB to 10 MB, preferably 0.5 MB to 1.5 MB, and the minimum partition size is in a range from 0.5 KB to 3 KB, preferably 0.5 KB to 1 KB. These encrypted pieces are then uploaded onto the network for distributed and/or decentralized storage (e.g., using SAFE Network) to be accessed at a later time by the data owner alone using their private encryption keys or by a party to whom the data owner has granted the requisite access (FIG. 2). The main hash that can call and piece back together the encrypted genome data fragments is stored on the genome data owner's account, and their encryption key is provided to only them. In this way, an individual's genome is secured against tampering and unauthorized access. It is worth noting that genomes have a natural hash map of sorts constituting of their genome references that are often used for alignment (e.g, human genome reference build 38), and as such, possession and decryption of all of the encrypted data pieces can be sufficient to reconstitute the genome data in its entirety.

The network handles genome preservation by keeping two or more copies of the same (encrypted) genome or segments of genome stored at random, distinct nodes across the network and across different geographical regions where nodes are spread. In some cases, an additional requirement can be implemented that requires the data to be spread across at least two continents or other geographically segmented areas. Furthermore, if a node storing a copy of the encrypted genome or segment of genome were to go offline or otherwise malfunction, the network automatically produces a new redundant copy from one of the remaining copies to replace the lost or unavailable copy. The storage location of these copies is continuously and automatically changing across the network so that they cannot be predicted. This unpredictable and extensive re-routing of encrypted genome data ensures that the stored genomes are preserved against natural disasters, human error or malice, and hardware malfunctions. The data can be periodically checked using checksums or cryptographic hash functions to verify its integrity during storage and transmission, allowing for self-repair using redundant copies if necessary.

Storage nodes herein described are computers, servers, or other data storage compute devices that are incentivized to participate in the storage layer of the network to host genomes. A reputation system can be utilized to ensure that more reliable nodes are entrusted with encrypted genome data packets over less reliable nodes. Consensus between nodes can be achieved using scalable consensus algorithms such as PARSEC (Chevalier et al., Maidsafe PARSEC Whitepaper. (2018)) or Hashgraph (Baird, Swirlds Tech Report SWIRLDS-TR-2016-01 (2016)) or spectrum-bound consensus where the scarcity of the electromagnetic spectrum can be leveraged for network protection through occupation by good actors. Furthermore, nodes can be organized in a scalable DAG or sharded DHT structure. Nodes are further incentivized against malicious behavior using various strategies including reputation, rating, age, and economic decentivization. The network could also be self-healing such that it can automatically restart and recover all data in the event of disruption. An existing public network with these characteristics (e.g., SAFE Network) can be utilized to this end with the relevant, herein described adaptations to handle the specific particularities of genome and omics datasets.

Though existing on the network, the genome data remains fully under the control of the data owner who may call the genome from any location in the world using their login credentials or private encryption keys. The data owner does not need to maintain storage but can outsource this importance function. Furthermore, the individual, the true data owner, has complete ownership of their data instead of any centralizing third party, and the data is heavily resistant to any hacking or other malicious behavior. Because the data is securely sustained on an ever evolving and persistent network, the genome is truly preserved for posterity. Safeguards can further be put in place to ensure that data owners can choose what happens to their data in the event of their death, e.g., entrusting their account keys to a family member, an estate planner, or a custodial service or maintaining their genome anonymously on network but homomorphically accessible to data seekers and service providers.

Performance features are enabled to ensure low latency access and processing of the data. Nodes can be assessed for network speed and other performance metrics, which are factored into the reputation of that node. In addition, the data can be compressed, in addition to being encrypted, to allow for faster transfer of data across the network.

The decentralized, server-less design, encryption, and continuous random routing of the encrypted data, all ensure that the data is highly resistant to hacking (e.g., DDoS, man in the middle, etc.). For instance, a DDoS or flooding attack would be cost-prohibitive for the attacker as they would have to pay to upload all of the data used in the attack to storage nodes. The attacker would also not know which node to attack as data storage is randomized and pieces are held across several nodes.

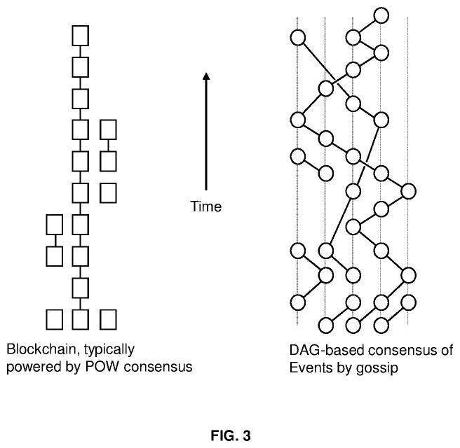

POW has become at risk of centralization since ASICS and high costs of mining have concentrated mining into the hands of a few conglomerates that can control and influence the popular blockchains if they so choose, a situation that blockchain was supposed to prevent. Other transaction confirmation mechanisms such as Proof of Stake do not decrease the risk for centralization, as larger nodes with more resources can have a disproportionate amount of influence on the network. Instead, using a DAG and gossip-based consensus algorithm such as PARSEC or Hashgraph, potentially supplemented with sharding where randomly chosen, reputable nodes can coordinate to secure the network, allows for network speeds of tremendously high throughput without compromising decentralization (FIG. 3).

To further decrease the possibility of centralization, nodes can be economically incentivized to perform their tasks based on various functions that scale differently with size of node or number of events handled. As nodes increase in size or number of events being processed, the economic incentives may increase rapidly up to a certain point and then tail off. For example, SAFE Network uses a sigmoidal rewards function to incentivize nodes of a certain size over others. Other similar functions may be used.

Accounts, IDs, and interfaces

Participants on the network will have accounts into which they log into using a unique alphanumeric key known to them alone or using biometric authentication. One account can be employed to manage a single genome (e.g., in cases where the participant is an individual managing their own genome alone) or multiple genomes (e.g., in cases where the participant is an organization or the participant is an individual managing their own genome as well as that of a significant other or pet). Optional provisions allow for the attachment to accounts of identity certificate hashes thereby allowing for regulatory compliance satisfaction for organizations that require it or for use in regions with Know Your Customer (KYC) or Anti-Money-Laundering (AML) laws.

Each account stores a hash to piece back together the fragmented, encrypted pieces of the account owner's genome stored across the network. When the data owner grants permission to a service provider or app to perform an operation on their genome, this hash is automatically and securely accessed without ever revealing it to the accessor. In other words, accounts are used for access control, identification, authentication, and authorization. Accounts are also used for many other purposes including value transfer (to receive rewards provided by a data seeker accessing the owner's genome or pay expenses charged by a service provider), settings of user profile and preferences, communications, negotiations, analyses, receipt of service results, etc.

To manage these functions while maintaining full privacy and security of the account owner, each account has an alphanumeric address that can be shared with other network participants. Accounts are anonymous by default but participants may have the ability to override this function. Further, an account can have one or multiple addresses associated with it. The addresses can further be made human-meaningful without compromising their security. The number of addresses associated with an account can increase or decrease over the lifetime of an account.

The network participant is always in full control over their accounts and associated addresses, thus of their identity and data thus preventing potential lock-in or lock-out by another party such as a service provider on the network. This setup further ensures that the data owner has the choice to employ the same address with multiple service participants (i.e., single sign-on) or a different address with each service provider.

A computer interface is employed to login into an account, and thus onto the network to perform actions (e.g., interact with genome or other omic data, place order, receive results, interact with network participants).

### Scale to Massive Arrays of Genomes and Other Omics Data

Access to large datasets of genomes can be critical in identifying the genetic features that underlie disease or other phenotypes. Examples of various costly efforts underway to sequence millions of people include those by Human Longevity (Weintraub, MIT Technology Review. (2016)) and Astra-Zeneca (Ledford, Nature. (2016)).

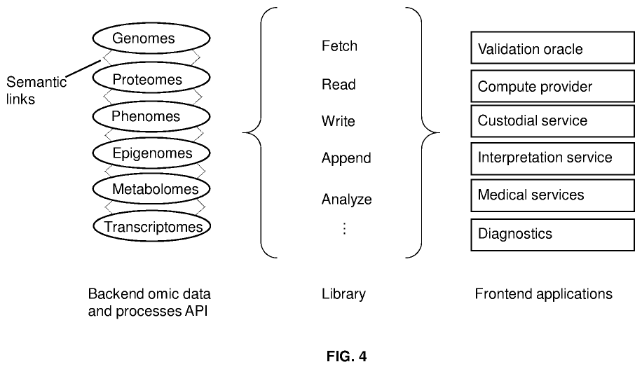

Links can be established between individual genomes to obtain large numbers of data on a scale never before possible to facilitate rapid identification of genetic features at the basis of disease or other phenotypes while still maintaining individual data owners' full privacy and ownership rights (FIG. 4). The specific links between genome data can be at the single base resolution or at lower resolutions (e.g., conserved regions). These links are anonymous and encrypted. The methods allow for genome data owners to have the option to make their data available to be linked to anonymously. They can also rescind that permission at any time without prejudice from the network. Links can be established automatically by the network or by service providers and oracles and can be read. Other oracles and network actors can confirm the links or dispute them, all without abridging the data owner's ownership, privacy or anonymity.

Such linking and integration can be achieved through many data linking protocols, for instance using the Resource Description Framework (RDF). RDF or the like can be further leveraged to tag specific portions of the genomes with helpful descriptive elements (e.g., provenance, citations, class, properties) that can be indexed for query using protocols such as SPARQL. RDF or the like can also be leverage for the integration of other heterogenous data (e.g., omics).

A further application that the methods provide for is linking potentially heterogeneous data to the genomes (or portions thereof) to automatically match their phenotypic information (e.g., how the data owner feels, photos of their ailments, how they sound, and data of their family) to their omics data to inform surveys, maps, and analyses. To ensure accuracy of information, the submission of this information may be performed by medical professionals, who may participate on network as oracles, and further validated by other oracles, as described below. The decentralized and secure integration of these seemingly disparate data can help provide insights to help the data owner's health or phenotypic outcome all while keeping their data private to only them and unblemished. Coupled with other aspects described herein (e.g., neural networks for analysis, outsource computation, oracle clusters, smart contracts for automation, etc.), a Semantic Web of the available genomes and omics data on the decentralized network can be established that is easily query-able as well as machine readable and provides for a very powerful means to accurately model the data owner digitally.

It can be anticipated that the powerful safeguards (privacy, preservation, and security) and incentives offered by the scalable genomes network described in the present invention should encourage many individuals and organizations to not only upload their genomes data onto the network but make it part of the genomes array, allowing for a genomic dataset of vast size and scale yet spread out cost that would otherwise be unachievable.

The thus obtained linked genome and omics arrays will allow for accurate, high resolution surveys, maps, and analyses to be performed within an organism (e.g., interactome), within a group (e.g., a specific human ethnic group), between groups (e.g., two or more breeds of the Canis familiaris species), between species (e.g., human vs chimpanzee), or across living organisms (e.g., mammals vs. insects or animals vs. plants) to unearth genetic features towards better understanding of life and development of solutions for better phenotypic outcomes.

Such a network of genomes and omics data, together with decentralized computing and AI-based analysis (vide infra) would allow for fine mapping of regulatory relationships in the cell where there are still great gaps, missing links, and false positives today. Further, the network allows for entire ecosystems to be simulated digitally.

Genome and omics data updates and requests would be implemented using webhooks and APIs.

Data linking methods can also involve artificial intelligence (e.g., neural nets) or other methods.

The network provides for database functionality without the requirement for use of databases (e.g., can employ Solid's semantic web structure which defines a web interface as well as a query language). Versatile, relational database query protocols such as SPARQL can be employed client-side or server-side. For non-relational database applications, protocols such as NoSQL can be employed. Decentralized database engines such as GUN can also be leveraged for graph-based engine allowing for real time data updates as well as offline synchrony.

Version control of genome and other omics data can be implemented to ensure referencing to the correct standard genome sequence or other control data. Recording data versions is especially important in the case of genome variant data, which must be referenced to the correct version of the appropriate reference genome. This reference information can be appended to the data and verified via oracles, as described below.

### Data Services Directly on Network for Convenience, Security, Privacy and Ownership

Certain participants on the network would provide genome and other omics data services to data owners including, but not limited to, analysis, interpretation, diagnostics, health counseling, and second opinions all performed privately and securely on the network and with the genome data owner always retaining full control of their data. There are several approaches to achieve this outcome where a service provider handles an owner's omics data without ever being able to breach their ownership or privacy rights. For instance, the data can be effectively analyzed within a homomorphic encryption state on network or otherwise without ever being unencrypted.

Furthermore, the identity of the data owner always remains known only to the data owner alone, ensuring full privacy during any exchange on the network, when providing data or acquiring services. Other parties such as those providing omics or network services can also maintain their identity private/anonymous. Still, parties providing services may have an incentive to reveal their identity on network so as to benefit from any existing brand image or goodwill. Alternatively, they can grow a brand value for their anonymous identity on network based on the validated effectiveness of their rendered services to network market participants over time. On network reputation and ratings systems would be employed for these purposes.

Data owners can also elect to make the results of any service available to a trusted third party (e.g., their healthcare provider).

Service providers' applications can run on user's devices (i.e., client side), on the service provider's own compute device (e.g., computer, server), or on network (on infrastructure contributed by a third party). Encrypted containerization can be used to segregate multiple data streams or processes and ensure anonymity or separation of network processes from non-related processes on that device.

The decentralized network in conjunction with the users' accounts would handle permissions and other processes typically handled by backend servers.

The present disclosure allows for API libraries to connect frontend service operations (e.g., analyzing omics data and displaying results) with backend processes of chunking, encrypting, storing, and routing the omics data.

### Freedom to Switch Between Service Providers

In existing centralized models, data service providers lock in users and make it extremely difficult if not impossible for them to port their data to another service. In the herein provided approach, the data owner's genome is never owned by a service provider. When the service provider accesses the data, it does so in a homomorphic encrypted state and the results of its query are either appended as a link to the data or taken with the service provider depending on the specific terms of data access that the owner agreed to.

In effect, the data ownership, privacy, and security remain unblemished, while the data owner can grant service providers permission to access it in anonymous state. If for any reason the data owner wishes to switch to another service provider or have multiple service providers perform on the data, they only need to revoke or grant access.

The permissions accorded to an application or service provider can be fine grained, i.e., ranging from access to the entirety of the data owner's library of genome, omics, description, and phenotypic data to access to only a subset of the data (e.g, exome portion of their genome).

Standard and common data formats would also be leveraged to ensure cross service and app access such that, for instance, another service could resume the incomplete work of another app.

Solid (Berners-Lee, solid.mit.edu. (2018)) is a framework that can be leveraged on the herein described network. Solid decouples applications from data produced allowing for data reuse and seamless switching between apps for users without loss of data or social connections.

Other frameworks for the Semantic Web such as the previously described RDF (Resource Description Framework) can also be leveraged for this achieve seamless integration and transition across data types, apps, systems and services on the described decentralized network. Per the W3C, “The Semantic Web provides a common framework that allows data to be shared and reused across application, enterprise, and community boundaries” (Hawke et al., W3C Semantic Web Activity (2013)).

### Artificial Intelligence for Processing, Analysis and Other Functions

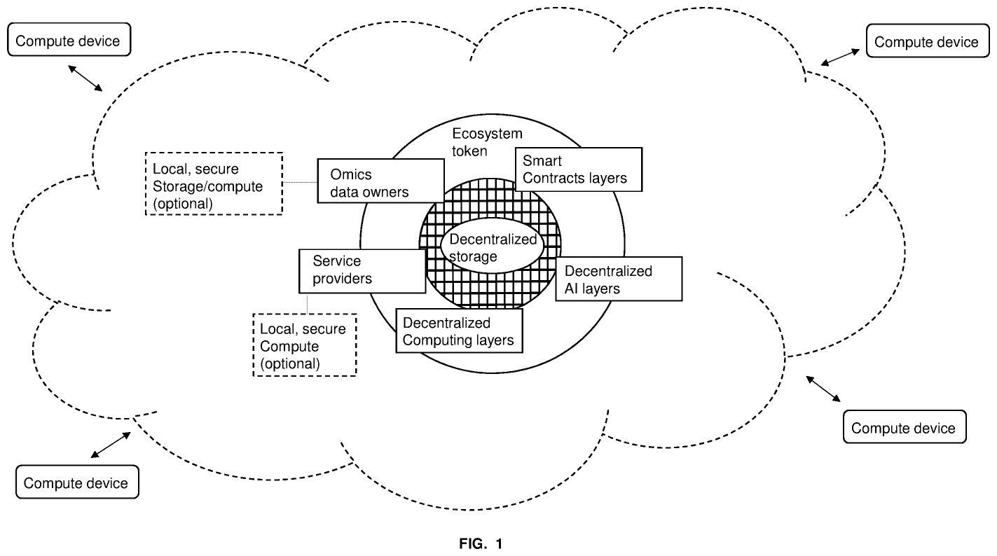

AI is ideally suited to the complexity of genomics and omics data processing, parsing, analysis, and interpretation. Various forms of AI can be incorporated for use on the decentralized network and by participants (FIG. 1). For instance, clustering algorithms (e.g., t-SNE, UMAP) could be employed to reduce dimensionality of the genome data and group data components into subsets: AI algorithms such as neural networks can be leveraged to identify and classify genetic features. Some examples of artificial neural networks and their healthcare applications include Baskin II et al., Expert Opin Drug Discov. 2016 August; 11(8):785-95 (2016); Hassabis D. et al., Neuron, 95(2):245-258 (2017).

Prior to deployment, training, validation, and testing of the AI models would be suitably performed off network as well as on network. In additional examples, the AI systems can be configured through backpropagation processes based on the training data.

The network itself could incorporate a decentralized, distributed AI mesh. Participating AI-based nodes on network can interact dynamically to achieve great scale and intelligent computing power. The system's architecture may provide for parameter specificities (e.g., number of layers if neural net, number of weights, values of the weights) or intelligent nodes can independently set their own AI parameters, providing more choice for service users.

These intelligent nodes would process omics data in real time and provide any number of services on network to data owners and seekers, as described above, in real time or otherwise. They can read, write, and memorize omics data, their topology, and relationships within and among data sets to provide actionable insights as to genetic and omics features and their effects. Useful training models and results can be automatically aggregated and shared among network participants.

An existing decentralized AI network can be leveraged, repurposed and incorporated on network for the herein described omics data processing and analysis (e.g., Satori. CognIOTA, SingularityNet). The network itself can also incorporate AI aspects directly, especially its components that are based on DAG-type structures, given that DAG lends itself well to the structure of a massively distributed neural network (e.g., Bayesian inference can be structured as a DAG: transaction weights connecting edges can be reimagined as the connection and training weights between neurons).

### Data Compression

While the scalable aspect of the network allows for the full genome or omics data to be encrypted and uploaded onto the network, the data may also be compressed to help save on storage costs and improve efficiency (e.g., upload speed). The data compression may occur prior to upload onto the network or it may take place on network as a service provided by a network participant.

Compression may involve any number of methods. General purpose compression protocols (e.g., gzip, 7zip) may be employed but specialized compression schemes that take advantage of the specific particularities of genomics and omics data can yield greater compression results. These include recording of the variations of the genome or omics data as compared to a specific reference sequence (i.e., variants), Burrows-Wheeler transformation (BWT) (Li et al., Bioinformatics (2009)), or encoding of the data into binary or trinary form or conversion into images through a matrix.

Usage of such compressed data could involve first decompression or direct usage in the compressed form without decompression (i.e., “compressive genomics” (Loh et al., Nature Biotechnology (2012)), depending on the deployed protocol (e.g., neural nets may employ the converted images of genetic sequences).

Such compression systems can further leverage AI programs for added efficiency and optimization.

### Agnostic Data Processing State

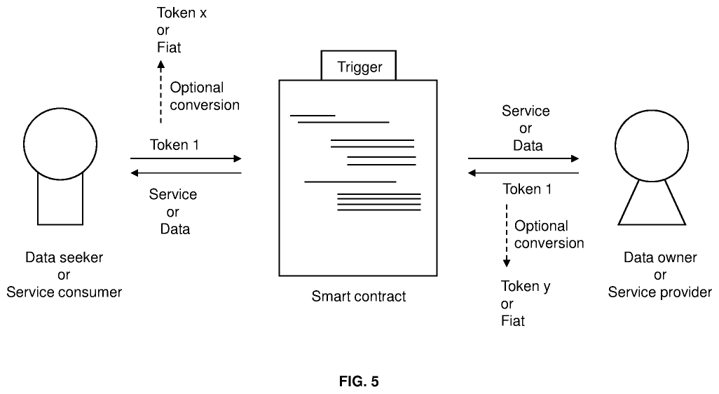

Genome or omics data can be uploaded to the decentralized network in any state of processing according to the designs of the data owner. For instance, in the case of genomes, the raw FastQ data could be uploaded to be automatically aligned and variant called prior to further analyses by service providers on the network using smart contracts (FIG. 5). More processed versions of the genome could also be uploaded such as a BAM file, CRAM file (compressed BAM), VCF file, etc.

While some may choose to conserve the VCF file alone in terms of storage costs, it is in fact tremendously advantageous to preserve the raw NGS FASTQ or SAM data because alignment and variant calling tools are continually improving, and with every improvement, it turns out that certain variants had been missed by older aligners and variant callers. These potential issues can be further remediated by the implementation of version control, mentioned above. In instances where storage is a concern, various compression solutions exist to reduce the size of raw sequence file as well as aligned sequence files.

### Oracles to Validate Data and More

Sometimes, machine or human error lead to incorrect data or information being provided. Malicious actors can also try to profit off fraudulent data (e.g., claim a rare mutation or upload the genome of a cat claiming that it is human). It is important to ensure that the omics data and associated information (e.g., phenotypes, analyses, results, etc.) uploaded onto the network are indeed correct. In centralized systems, the central authority carries out this validation function. On the decentralized genome network, this validation and verification function can be accomplished through the use of oracles.

Oracles would be a type of service provider on the network whose principal purpose is to verify data correctness. Oracles are employed to autonomously upload or confirm and validate the characteristics of the genome or other omics data (e.g., correct reference version, completeness of genome, coverage, species, etc.) and their descriptive appended information (e.g., phenotypic information of genome data). Oracles in this case could be the persons or institutions (e.g., medical professional) that performed the tests or generated the data for the data owner or oracles can be automated analyses tools (e.g., machine learning based) that identify specific markers in the data to validate the appended descriptive information.

Oracles can also validate results provided by other service providers as well as verification results provided by other oracles. In the latter case, an omics data owner or data seeker may call upon several oracles to perform a validation such that if a majority (i.e., quorum) of oracles supply the same results, then the oracle service requestor has greater confidence that the validation results are accurate.

As with other service providers, oracles may choose to be anonymous or reveal their identity (e.g., to benefit from off network brand value). In either case, an oracle's reputation, which is based on a combination of several metrics, such as the accuracy of their provided services, reliability or uptime, or user-based rating system, will affect how often that oracle is called upon by service seekers.

Oracles can offer their service for free or for a fee. The fee can be based on factors such as the verified accuracy of the service result, the turnaround time, or the availability of the service to be provided by one or more separate oracles who may be competing to complete the same task.

Oracles in the herein described network could be implemented through any number of methods (e.g., ChainLink, IOTA's Qubic network, etc.).

### Groups of Service Provider Nodes to Increase Omics Analysis Accuracy

Service providers (including oracles) may join in groups to render services to data owners and seekers or to the network (FIG. 1). This type of arrangement would help increase the correctness of the genome data analysis given that a majority of participating service providers could output the same results for the analysis to be taken as correct, thus recorded by the network and delivered to the service requestor. It would also reduce the impact of potential malicious actors or erroneous results.

Genome data service seekers may also select the number of service providers that they would like to perform the same task toward ensuring that the results are accurate based on quorum of service providers (e.g., greater number of participant service providers for critical tasks). In some instances, one service provider with a longstanding, stellar reputation may provide results whose accuracy is trusted by the network, but generally, the majority results provided by a group of service providers would be more trusted over the result provided by a single service provider with low reputation.

In some cases, different service providers can work on separate subtasks and link together the results afterwards to complete the requested task. This parallelization of the task can be combined with establishing a quorum for each individual subtask among a group of service providers.

There are several methods to achieve on network groups of service providers (e.g., Ethereum, Safe Network compute layer, IOTA's Qubic, etc.).

### Smart Contracts for Trustless Service Exchanges and Increased Automation

Smart contracts would be employed on network to automatically execute agreements between participants on the network such as storage, validation, analyses, negotiation, distribution, marketing, etc. (FIG. 1. FIG. 5). For instance, when omics data is uploaded onto the network, smart contracts can automatically identify and pay oracles for validation of the data without the data owner having to be manually and repeatedly be involved. Further, smart contracts can handle the marketing and access to the data and automatically adjust prices based on the market rates on the network. If there's an attractive network market rate for an analysis of the data that could be of interest to the data owner, the smart contract can notify and automatically carry out the transaction to obtain the analysis. Once a smart contract is activated, the service provider can automatically perform the analyses and submit the results.

The smart contract protocol can further integrate automatic management of participant account balances, e.g., check whether the service seeker account has enough funds to cover the service fee before activation or can take remedial actions such as ordering a service on credit.

Smart contract protocol can be implemented directly within the network or leverage existing, public smart contract protocols (e.g., Ethereum. Safe Network, IOTA Qubic).

### Decentralized Computing Layer

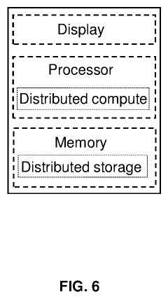

The great heterogeneity and enormity of genomes and other omics data have translated to enormous computational demands to process and analyze them. This reality has introduced a centralization component as the organizations with the greatest computing resources attract most of the omics data with which they are entrusted (FIG. 1, FIG. 6).

The herein described decentralized network provides for decentralized computing that removes the inherent risks of centralization described above. Furthermore, the decentralized computing layer ensures that all researchers and private individuals have the same access to great computing resources yet their data remain fully their own and never at risk of control by a centralizing party. For instance, blockchain miners on Bitcoin produced over 50,000,000 hash rate of computational power in August 2018 (www.blockchain.com/en/charts/hash-rate (2018)), which all went to solving meaningless cryptographic puzzles per Bitcoin's current design. On the described genome network, the consensus mechanism of the network itself is virtually costless compared to POW. Instead, this type of enormous computing power from miners who would no longer need to solve these cryptographic puzzles for POW could be leveraged to solve real problems (e.g., provide computing power to oracles, participate in an network AI program for omics data analysis), and hash power-providers would still be rewarded for providing the computational power.

Network participants may outsource computation intensive tasks to computing nodes on the network in fully trustless fashion. Parallelization protocols (e.g., MapReduce) may be leveraged to spur parallel computing and reduce bandwidth bottlenecks. In certain embodiments, homomorphic encryption is employed to query, compute, and derive insights from encrypted private data without breaking data owner's privacy or anonymity.

This computation layer can be native to the network or supplied from an existing network (e.g., Ethereum, Safe Network compute layer. IOTA Qubic computation layer).

### Network Token and Ledger to Facilitate the Exchange of Data and Services

The network would comprise of a token to facilitate and incentivize the exchange of genome data and services described above (FIG. 1, FIG. 5). The token is secure, highly scalable (i.e., transaction speeds greater than thousands per second to instant), and feeless (i.e., if one token is sent, one token is received without any other fee to facilitate the transmission). Token transactions constitute a type of event that can be processed and recorded by network nodes.

Given its scalability and feeless properties, the token further provides for very granular data consumption and payment (e.g., micropayments). For instance, a data seeker could stream only a certain portion of a genome and pay only for that accessed portion at an example rate 0.1 cent per base.

The network token is further exchangeable to other valuable network tokens (e.g., BTC, ETH, SAFE) or to fiat currencies (e.g., USD, Euro). Libraries can be integrated for conversion to occur automatically on network.

The network further provides for participants to offer their data or services for free. Other incentivization models would include a data seeker offering to provide a service in return for homomorphic encrypted data access or another different service.

### Autonomous Marketplace for Omics Data and Services

In certain implementations, the network is fully autonomous with no human intervention to determine the cost of storage or services (FIG. 1, FIG. 5). Rather, prices are set automatically by the network based on calculated resource availability and demand across the decentralized ecosystem. Economic incentivization and decentivization contribute to good citizenship and good faith actions by all network participants.

Network participants would be provided the option to override the price set by the network and provide their own. They can also form various dynamic economic clusters to boost efficiency, security and productivity.

In some instances, bundling of services or data can be done autonomously. For example, the network can determine groups of similar data that would be useful for a certain application and provide the data together or separately to a requesting network participant granted appropriate permissions from data owner.

Allocation of computing power for various services can also be automated. Certain nodes or systems may have excess computing power that they wish to provide to the network but are agnostic to the type of service being provided. The network could autonomously tap into that excess computing power when a service is requested and create contracts between service providers and requestors. In some instances, another service provider or oracle could provide or assist in third party management of such a process.

### Additional Omics Data to Supplement Genomes on the Network

As mentioned in certain parts of the present disclosure, different omics data sets (e.g., kinome, proteome, phenome, transcriptome, interactome, epigenome, microbiome, metabolome, etc.) can be uploaded to the network to be stored, processed, validated, analyzed, integrated, linked, or marketed in a synergistic fashion or otherwise to the genomics datasets. These data sets can be linked or unlinked to one another in whole or in parts.

### Other Living Species

In addition to H. sapiens, the network provides for genomics and omics data from any living or extinct species (e.g., H. erectus, bacteria, mouse, worm, insects, domestic pets, etc.) to be uploaded to the network to achieve any number of purpose, including storage, processing, validation, analysis, or marketing.

### Signaling and Settlement Component

In another implementation, the decentralized sharing of genomes is achieved through a signaling and settlement approach where the signaling (i.e., initial connection and negotiation) between the exchanging parties is accomplished through a secure, decentralized network (e.g., SAFE Network, Ethereum, IOTA's Tangle network) and upon the parties coming to agreement on the exchange or service terms, the data is transferred on a direct peer-to-peer basis from the genome or other omics data owners' compute device to that of the genome or other omics data users through a settlement layer (e.g., WebRTC library) without any intervening centralizing third party.

Such an option would be provided on network for certain parties that might find it useful for their specific use cases. For instance, two companies that want to exchange omics data that they are storing locally could establish a decentralized connection, agree on terms, and proceed to sharing the data directly without first moving it to the full network potentially to save on cost of network storage (e.g., if they already have sunk costs from their own local storage).

In these cases, the herein described decentralized network serves as a secure signaling and negotiation meeting place to coordinate and establish secure peer-to-peer connections directly between exchanging parties. The participants can then employ computation and analysis resources off network or leverage resources from the network to supplement their own infrastructure where there are gaps.

### Additional Detail on Implementation

Implementation of the innovations disclosed herein is obvious to one skilled in the arts.

For instance, it is obvious to one skilled in the arts that nodes in the decentralized network are compute devices (FIG. 6), and that access to and operations on the decentralized peer-to-peer network can also be achieved through compute devices. In one embodiment, such compute devices are used to interface with the decentralized peer-to-peer network.

Identities on the network can be derived from a key pair system. In one embodiment, a private or secret key known only to the participant party from which a number of public keys can be deduced, authenticated by digital signature, and serve as public identities. Public identities on the network can thus be cryptographically assured. Participating parties then have the option to attach their legal identity to any of their public identities. An example of key pair generation is by the well-established public-key cryptography system ECDSA algorithm, which is a PGP program.

In one embodiment, a transaction between two or more participating parties on the network is carried out securely using these public identities. Data access can be granted using permissions depending on the specific agreement between the parties and implementation.

In one specific embodiment, access can be granted by permanently transferring ownership of the data (e.g., through transfer of access token, ownership key transfer, or transfer of data map of encrypted file).

In another specific embodiment, access can be granted by transferring ownership (temporary or permanent) of the encrypted data, which the accessing party could analyze using homomorphic encryption protocol (e.g., HElib, PALISADE, FHEW, Duality, STARK, zksnarks, etc.) and return the thus encrypted result back to the original data owner, who can then decrypt it using their private key and thus ensuring that the results are known to only them and not even to the party performing the analysis. In one specific implementation, the data owner can also change the ownership of the encrypted results, if agreed upon, thus allowing the accessing party to decrypt the results alone. In such implementation, the original input data remains the sole property of the data owner.

In another specific embodiment, access can be granted by granting permissions for the location of the encrypted data to be temporarily changed to an agreed upon compute device that will guarantee the original attributes of the data as well as its ownership even as analysis is performed on it (e.g., in a secure enclave; Intel SGX is an example of a secure enclave).

In one embodiment, a smart contract can be used to facilitate these transactions (FIG. 5). In a specific embodiment, the smart contract can be a basic script that initiates certain actions when certain conditions are met. In a specific embodiment, a multi-sig algorithm such as a Boneh-Lynn-Shacham (BLS) can be employed in this regard to efficiently ensure that the initiated action is based on the provable input of a majority of participating parties in the smart contract. Examples of process logic include, but are not limited to:
(1) if signature by data owner, change data attributes (e.g., ownership, location, etc.) according to instructions from other transacting party;
(2) if condition met (e.g., other transaction party meets set price, signature from majority of multi-sig, etc.) permission X (e.g., ownership, location transfer, data modification, etc.) is granted to provided key;
(3) if condition met (e.g., analysis or interpretation data is appended to omics data), transfer Y amounts of coins to key provided by party that supplied analysis data.

In certain embodiments, to connect, operate, and transact on the network, an interface is employed. The interface can be a command line interface (CLI) that is primarily text-based or a graphical user interface (GUI) that is based on graphical icons and visual indicators. It is through this interface that participating parties on the network manage permissions, make requests, transact, set smart contracts, etc. Furthermore, this interface can be implemented within a dedicated standalone program or through a web browser where access would be by navigating to certain link. The interface can further incorporate a dashboard that provides information to a user including: which data they have on network, what permissions they have granted, how much money or token they have on their balance, which data they have been granted access to, how much storage they are occupying, notifications of messages from other parties, etc.

In one embodiment, connections, communications and transfers between parties can be routed using transport network protocols. QUIC is an especially well suited one given its advantages compared to protocols such as TCP. A well-suited peer-to-peer implementation of QUIC is quic-p2p in the Rust programming language.

In certain embodiments, when a user (data owner) attempts to upload omics data onto the network, they would be presented with the option to append tags to the data to allow for easy recognition and cataloguing. Further, these tags can be encrypted or unencrypted. Examples of information that these tags may record include price if any for access by other parties, data type (e.g., genome, transcriptome, proteome, etc.), file type (e.g., bam, vcf), data quality (e.g., coverage level), species (e.g., human, dog), age of subject, phenotype of subject (e.g., disease, physical attribute), model and make of sequencer machine used to produce the data (e.g., Illumina NovaSeq 6000), protocol followed to process the data (e.g., GATK best practices), type of alignment for genomes (e.g., de novo, reference-based), reference genome version for genomes (e.g., human GRCh38), owner's preferences and licenses (e.g., access rules, study types that the data can be used for, organizations that are blacklisted from access), permissions (e.g., comments can be appended after access describing data quality), etc.

In yet other embodiments, algorithms can be employed to crawl the network to identify and catalogue these omics data tags. Thereby, a party in need of a specific type of omics data can more readily find them at a later time and ascertain certain information about the data to determine whether they want access without needing to access or decrypt the actual data.

While this invention is satisfied by aspects in many different forms, as described in detail in connection with the preferred invention, it is understood that the present disclosure is to be considered as example of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. All references cited herein are incorporated by their entirety for all purposes. In the claims that follow, unless the term “means” is used, none of the features or elements recited therein should be construed as means-plus-function limitations pursuant to 35 U.S.C. § 112, ¶6.

What is claimed is:

1. A method executed on a processor for storing omics data that indicates long sequences of elements associated with a particular biological molecule, the method comprising:
receiving digital omics data comprising over two kilobytes (KB, 1 KB=1024 bytes, each byte equal to 8 bits);
splitting the digital omics data into a plurality of partitions, each partition comprising a number of elements in a sequence of the omics data, wherein the number of elements is not greater than a maximum partition size, and the maximum partition size is much less than then a number of elements in a typical instance of the particular biological molecule;
encrypting each partition to form an encrypted partition;
inserting each encrypted partition into a corresponding data packet that includes an owner field that holds data that uniquely indicates an owner of the omics data; and
uploading each data packet into a non-centralized, peer-to-peer distributed storage network.

2. The method of claim 1, wherein the maximum partition size is in a range from 0.5 megabytes (MB, 1 MB=1024 KB) to 10 MB and preferably in a range from 0.5 MB to 1.5 MB.

3. The method of claim 1, wherein the number of elements in each partition is not less than a minimum partition size and the minimum partition size is in a range from 0.5 KB to 3 KB and preferably in a range from 0.5 KB to 1 KB.

4. The method of claim 1, wherein the owner field is encrypted.

5. The method of claim 1, wherein the corresponding data packet includes a token field that holds data that indicates a means to pay for a service that operates on the encrypted partition.

6. The method of claim 1, wherein the corresponding data packet includes a sequence field that holds data that indicates a position of the encrypted partition in the data packet relative to a different partition in the digital omics data.

7. The method of claim 6, wherein the sequence field is encrypted.

8. The method of claim 1, wherein the omics data includes data about one or more biological molecule types selected from a group comprising genomes, proteomes, kinomes, phenomes, epigenomes, metabolomes and transcriptomes.

9. The method of claim 1, wherein each data packet is stored on a plurality of different nodes on the distributed storage network.

10. The method of claim 9, wherein at least two different nodes of the plurality of different nodes on which each data packet is stored are in two different geographical regions.

11. The method of claim 1, further comprising compressing each partition before said encrypting.

12. The method of claim 1, further comprising compressing each encrypted partition after said encrypting.

13. The method of claim 1, wherein the corresponding data packet includes a metadata field that holds data that indicates descriptive information about the partition.

14. The method of claim 13, wherein the metadata field is encrypted.

15. The method of claim 1, wherein said encrypting is homomorphic encrypting relative to one or more operations on the encrypted partition.

16. The method of claim 1, further comprising granting permission to a second party different from the owner of the omics data to decrypt a field in the data packet.

17. The method of claim 16, wherein said granting permission to the second party is performed in response to receiving a payment.

18. The method of claim 17, wherein the payment is in a form of a network token.

19. The method of claim 16, further comprising receiving an analysis result based on the field decrypted in the data packet in response to granting permission to the second party.

20. The method of claim 19, wherein said receiving an analysis result is performed in response to sending a payment.

21. The method of claim 20, wherein the payment is in a form of a network token.

22. The method of claim 16, wherein said granting permission to the second party is facilitated automatically using a smart contract.

23. The method of claim 19, further comprising inserting, into an analysis field in the data packet, data that indicates the analysis result.

24. The method of claim 1, wherein a catalogue of available omics data from one or more owners of the omics data is maintained on the distributed storage system.

25. The method of claim 16, wherein a plurality of digital omics data from one or more owners of the plurality of digital omics data are used by the second party.

26. The method of claim 19, wherein said encrypting is homomorphic encrypting relative to one or more operations on the encrypted partition and the analysis result is obtained without decrypting the data packet.

27. A non-transitory computer-readable medium carrying one or more sequences of instructions, wherein execution of the one or more sequences of instructions by one or more processors causes the one or more processors to perform the following:
receive digital omics data that indicates long sequences of elements associated with a particular biological molecule, said omics data comprising over two kilobytes (KB, 1 KB=1024 bytes, each byte equal to 8 bits);
split the digital omics data into a plurality of partitions, each partition comprising a number of elements in a sequence of the omics data, wherein the number of elements is not greater than a maximum partition size, and the maximum partition size is much less than then a number of elements in a typical instance of the particular biological molecule;
encrypt each partition to form an encrypted partition;
insert each encrypted partition into a corresponding data packet that includes an owner field that holds data that uniquely indicates an owner of the omics data; and
upload each data packet into a non-centralized, peer-to-peer distributed storage network.

28. An apparatus comprising:
at least one processor, and
at least one memory including one or more sequences of instructions,
the at least one memory and the one or more sequences of instructions configured to, with the at least one processor, cause the apparatus to perform at least the following,
receive digital omics data that indicates long sequences of elements associated with a particular biological molecule, said omics data comprising over two kilobytes (KB, 1 KB=1024 bytes, each byte equal to 8 bits);
split the digital omics data into a plurality of partitions, each partition comprising a number of elements in a sequence of the omics data, wherein the number of elements is not greater than a maximum partition size, and the maximum partition size is much less than then a number of elements in a typical instance of the particular biological molecule;
encrypt each partition to form an encrypted partition;
insert each encrypted partition into a corresponding data packet that includes an owner field that holds data that uniquely indicates an owner of the omics data; and
upload each data packet into a non-centralized, peer-to-peer distributed storage network.

29. A system comprising the apparatus of claim 28 and a plurality of peer-to-peer nodes organized in a distributed storage system network.
